(19) United States
(12) Patent Application Publication
(10) Pub. No.: US 2002/0082697 A1
(43) Pub. Date: Jun. 27, 2002

(54) Implantable osteogenic material

(76) Inventor: Christopher J. Damien, Denver, CO (US)

Correspondence Address: Timothy L. Scott, SULZER MEDICA INC., Suite 1600, 3 East Greenway Plaza, Houston, TX, 77046-0391, US

(21) Appl. No.: 09/747,038

(22) Filed: Dec. 22, 2000

(51) Int. Cl. A61F 2/44; C07K 1/00

(52) U.S. Cl. 623/17.16; 530/356; 424/423

(57) ABSTRACT

Improved collagen-based osteogenic materials are disclosed that have improved shaping and handling properties and which are easier for the health care practitioner to use than conventional implantable osteogenic materials. The new shaped implantable compositions or devices also provide a good matrix for the release of osteogenic substances and other desirable biologically active agents at the site of implantation to promote bone growth.

### BACKGROUND OF THE INVENTION

1. Field of the Invention

The present invention generally relates to the growth or regeneration of bone in the body. More particularly, the invention relates to collagen-containing osteogenic compositions, their manner of making and to methods of using the compositions to induce in vivo osteogenesis.

2. Description of Related Art

A variety of implantable materials have been used in the delivery of active compounds such as growth factors to a patient. One area of investigation that is currently receiving substantial interest is the development of implantable materials that can be used in the repair of bone injuries and defects. Typically, these materials are implanted at a desired site to promote osteogenesis. Ideally, such a material should have the ability to adhere and conform to the implanted site and facilitate bone growth, and to deter overgrowth of non-bone tissue in the implant site, and to be immunologically tolerated by the host, and to serve as a framework for the newly forming bone tissue.

U.S. Pat. Nos. 5,314,476 and 5,073,373 disclose a deformable, shape-sustaining osteogenic composition comprising demineralized bone particles and a polyhydroxy compound such as glycerol, or an oligosaccharide. It is known that glycerol at certain levels can be toxic.

U.S. Pat. Nos. 5,405,390 and 5,236,456 disclose a surface-adherent osteogenic composition derived from demineralized and thermally modified bone tissue. The composition is administered in the form of a powder, a viscous liquid, or by direct injection.

U.S. Pat. No. 5,246,457 discloses a bone-repair composition comprising a calcium phosphate salt and reconstituted fibrillar atelopeptide collagen. It does not include any biologically active ingredients. The physical and handling properties are said to be improved by a number of curing processes, including heat, maturation of the wet mixture and/specific cross-linking of collagen.

U.S. Pat. No. 4,440,750 discloses an osteogenic composition comprising demineralized bone powder and reconstituted native atelopeptide collagen fibers in a continuous aqueous phase having a substantially physiologic pH and ionic strength. U.S. Pat. No. 4,394,370 discloses a bone graft material for treating osseous defects, the material comprising collagen and demineralized bone particles. A mineralized collagen bone grafting matrix, which may include a bone growth factor, is described in U.S. Pat. No. 5,776,193.

Existing osteogenic compositions have relatively poor handling characteristics. Thus, when a surgeon attempts to reconstitute and implant such compositions, there is considerable difficulty in properly handling and/or implanting the material at the desired site within the body. Because of these poor handling characteristics, an implant that may be optimally configured for one site may prove difficult or impossible to implant at another site. Accordingly, if the surgeon 15 finds during surgery that the optimum delivery site is different from what was anticipated prior to surgery, the surgeon is faced with the choice of either implanting the material at a sub-optimal location or risk failure of the material by implanting it at a site for which it is ill-suited. In extreme cases, it may not be reasonable to place the composition in any site. Health care practitioners frequently observe subsequent loss or displacement of the implanted material from the implant site before sufficient time has elapsed for new bone to form at the site.

### SUMMARY OF THE INVENTION

The present invention seeks to overcome these and other limitations inherent in the prior art by providing methods, compositions, devices and kits for in vivo repair or restoration of osseous defects. Accordingly, the invention relates to implantable osteogenic compositions and materials with superior shaping and handling properties, making the compositions easier for the health care practitioner to deliver to a desired physiological target site than conventional implantable osteogenic materials, which are typically only suitable for use in fixed-configuration implant sites. The compositions and materials also provide superior in vivo delivery of the osteogenic growth factors comprising part of the new compositions. In another embodiment, the new implantable compositions or devices also provide a good carrier and delivery system for the optimal release of osteogenic substances and other desirable biologically active agents at the physiological target site to promote bone growth. In a preferred embodiment, the compositions of the present invention are capable of compression and expansion to fill a defined defect or cage site. Likewise, depending on the site, certain of the compositions can be compressed to fill odd-shaped defects and expanded upon contact with body fluids after implantation. In many cases the compositions act as scaffolds for new bone formation.

In accordance with certain embodiments of the present invention, a method of making an osteogenic composition is provided. The method comprises combining purified collagen with water containing dilute acid and a predetermined amount of an osteoinductive substance. These ingredients are placed in a dispersion assembly comprising two vessels coupled by a narrow channel or reduced diameter portion to establish fluid communication between the vessels. In a preferred embodiment the vessels comprise syringes that joined together by a coupler fitting, such as a Luer type adapter. The combined ingredients are extruded through the coupler from one vessel to the other a predetermined number of times by passing the dispersion back and forth from syringe to syringe, through the reduced diameter portion, causing the collagen to hydrate and producing a partially thickened mixture or dispersion. The partially thickened mixture is allowed to stand for a time, preferably about 30-60 minutes, more preferably about 60 minutes, to permit additional thickening. In certain preferred embodiments, the mixture is again passed from vessel to vessel a predetermined number of times, resulting a still more viscous gel-like mixture. This mixture is allowed to stand for a longer time interval, preferably overnight, such that a thick, extrudable mixture or dispersion is obtained. The thickened mixture is then extruded, preferably into a mold, and dried to obtain a dehydrated osteogenic matrix material suitable for packaging for later rehydration and implantation by a surgeon.

In one embodiment, the dispersion comprises approximately 1-8% (wt./vol.) collagen, which is preferably fibrous mammalian tendon type I collagen in 10 mM HCl, or another dilute acidic solution. The dehydrated matrix material may also be sterilized prior to packaging and shipment. In some embodiments, the dehydrated product is subsequently rehydrated. For example, in a surgical setting the product can be conveniently removed from the packaging and rehydrated by the surgeon immediately prior to implantation. The rehydrated osteogenic matrix may optionally be mixed with a bulking agent to provide a shapeable osteogenic paste-like material for implantation. In one embodiment, the bulking agent comprises demineralized bone matrix particles. The paste-like material may also be shaped into a desired configuration before implanting into a target physiological site. In another embodiment, the rehydrated product is compressible and can be easily inserted into a spinal cage to provide an implantable osteogenic device for use in spinal fusion procedures. In some embodiments the osteoinductive substance in the composition is a bone growth protein, bone morphogenetic protein 1-13, osteogenic protein-l or 2, FGF-1 or -2, TGF-beta, or GDF-5,6 or 7.

In accordance with certain other embodiments of the present invention a method of making an implantable osteogenic device that contains an above-described osteogenic composition is provided. In some embodiments the method of making includes shaping the composition into a desired three dimensional configuration. In one embodiment the method includes inserting the composition into a spinal cage, or other such implantable device and an improved osteogenic spinal cage is provided.

Still another embodiment of the present invention provides a method of inducing osteogenesis in a subject in need of therapeutic treatment. One of the above-described devices is implanted at a site in the body where osteogenesis is desired. In certain embodiments the site is a dental or periodontal defect, and in certain other embodiments the site of implantation is the intertransverse process space between two vertebral bodies that are desired to be fused together.

Also provided according to certain embodiments of the invention is a method of making a collagenous matrix. The method includes combining collagen and a dilute aqueous acid solution in a dispersing assembly as described above. The method also includes passing the combined ingredients from a first vessel to a second vessel through a narrow channel or tube a predetermined number of times to subject the ingredients to physical forces sufficient to disperse the collagen in the water. In the process, the collagen becomes at least partially hydrated and an extrudable dispersion results. The thickened dispersion is allowed to stand for a predetermined time interval such that a thick, extrudable dispersion is obtained. In some embodiments this extrudable dispersion is placed in a mold and dried. In some embodiments the dried matrix is rehydrated and may be mixed with a bulking material. In some embodiments a biologically active agent is included with the collagen mixture. Collagen preferably comprises about 1-8% (wt./vol.) of the dispersion.

A method of administering a biologically active agent to a subject in need of treatment is also provided according to some embodiments of the present invention. A delivery vehicle comprising the above-described collagenous matrix containing a biologically active agent is implanted at a target site in the body of the subject. This may be done surgically or by injection.

The biologically active agent, which could be a pharmaceutical agent or drug, is then allowed to be released from the delivery vehicle at the site of implantation. These and other embodiments, features and advantages of the present invention will become apparent with reference to the following description.

### BRIEF DESCRIPTION OF THE DRAWINGS

For a more detailed description of the present invention, reference will now be made to the accompanying Figures, wherein:

FIG. 1 illustrates an SDS-PAGE of one embodiment of the present angiogenic protein mixture, both in reduced and non-reduced forms;

FIG. 2 is an SDS-PAGE gel of HPLC fractions 27-36 of a protein mixture according to an embodiment of the present invention.

FIG. 3 is an SDS-PAGE gel with identified bands indicated according to the legend of FIG. 4;

FIG. 4 is an SDS-PAGE gel of a protein mixture according to an embodiment of the present invention with identified bands indicated, as provided in the legend;

FIG. 5 is a two dimensional (2-D) SDS-PAGE gel of a protein mixture according to an embodiment of the present invention with internal standards indicated by arrows;

FIG. 6 is a 2-D SDS-PAGE gel of a protein mixture according to an embodiment of the present invention with circled proteins identified as in the legend;

FIGS. 7A-O are mass spectrometer results for tryptic fragments from one dimensional (1-D) gels of a protein mixture according to an embodiment of the present invention;

FIG. 8 is a 2-D gel Western blot of a protein mixture according to an embodiment of the present invention labeled with anti-phosphotyrosine antibody;

FIGS. 9A-D are 2-D gel Western blots of a protein mixture according to an embodiment of the present invention, labeled with indicated antibodies.

FIG. 9A indicates the presence of BMP-3 and BMP-2.

FIG. 9B indicates the presence of BMP-3 and BMP-7.

FIG. 9C indicates the presence of BMP-7 and BMP-2, and FIG. 12D indicates the presence of BMP-3 and TGF-β1;

FIG. 10 is a PAS (periodic acid schiff) stained SDS-PAGE gel of HPLC fractions of a protein mixture according to an embodiment of the present invention;

FIG. 11 is an anti-BMP-7 stained SDS-PAGE gel of a PNGase F treated protein mixture according to an embodiment of the present invention;

FIG. 12 is an anti-BMP-2 stained SDS-PAGE gel of a PNGase F treated protein mixture according to an embodiment of the present invention;

FIGS. 13A-B are bar charts showing explant mass of glycosylated components in a protein mixture according to an embodiment of the present invention (FIG. 13A) and ALP score (FIG. 13B) of the same components;

FIG. 14 is a chart showing antibody listing and reactivity;

FIGS. 15A-B together comprise a chart showing tryptic fragment sequencing data for components of a protein mixture according to an embodiment of the present invention;

FIGS. 16A-F together comprise a chart showing tryptic fragment mass spectrometry data for components of a protein mixture according to an embodiment of the present invention;

FIGS. 17A-B are an SDS-gel (FIG. 17B) and a scanning densitometer scan (FIG. 17A) of the same gel for a protein mixture according to an embodiment of the present invention;

FIG. 18 is a chart illustrating the relative mass, from scanning densitometer quantification, of protein components in a protein mixture according to an embodiment of the present invention; and

FIGS. 19A-D together comprise a chart showing mass spectrometry data of various protein fragments from 2D gels of a protein mixture according to an embodiment of the present invention.

### DETAILED DESCRIPTION OF PREFERRED EMBODIMENTS

It has been previously discovered that an osteogenic collagen-based composition can be made easier to handle by the surgeon and more readily kept in place in the implant site by adding certain acids to the composition, as described in co-assigned U.S. Patent App. Ser. No. 09/023,612 entitled “Implantable Putty Material.” The entire disclosure of that application is incorporated herein by reference. In the presently disclosed studies, the inventor has devised new collagen-based osteogenic compositions with improved structure and handling characteristics that are prepared using special manipulation techniques. The new compositions have highly desirable physical properties such as improved cohesiveness, elasticity and the ability to be molded to a desired shape, that are at least equal to, and in most cases are superior to those of the compositions disclosed in U.S. Patent App. Ser. No. 09/023,612.

In general, the new compositions are prepared by mixing collagen, a dilute aqueous acid solution and an active ingredient, preferably a bone growth factor using a special processing procedure that results in an osteogenic composition or solid device with improved handling characteristics, that holds a desired shape better than conventional osteogenic compositions, permitting the shaped composition, or device, to stay in place in an implant site (e.g., a dental socket) better than other compositions or devices. Representative compositions and their use in periodontal and spinal fusion applications, are described in the Examples that follow.

General Materials and Methods

Collagen.

Preferably purified fibrillar bovine tendon type I collagen is obtained from Regen Biologics, Redwood City, Calif. Alternatively, fibrillar collagen, atelopeptide collagen, telopeptide collagen or tropocollagen can be collected from a variety of mammalian sources. Methods for preparing atelopeptide collagen and tropocollagen are described by Glowacki et al. in U.S. Pat. No. 4,440,750, which is incorporated herein by reference. Preferably the amount of collagen present in the new osteogenic materials and compositions is from about 1-10 % (by weight), more preferably from about 1-8%, or as specified in the examples that follow.

Osteoinductive Substances and Other Active Agents

An “active ingredient” or “biologically active agent” refers to any compound or mixture of compounds that have a biological activity. Exemplary active ingredients include osteoinductive substances, growth factors, hormones, antibiotics, anticancer agent and antiviral compounds. Osteoinductive substances are described in detail below. Growth factors can include fibroblast growth factor (FGF-1 or 2) and transforming growth factor beta (TGF-beta) (See Cuevas et al., “Basic Fibroblast Growth Factor (FGF) Promotes Cartilage Repair in vivo,” Biochem Biophys Res Commun 156: 611-618, 1988) These growth factors have been implicated as cartilage stimulating and angiogenic agents. FGF-1 or 2, for example, has been shown to increase the rate of osteoblast replication while simultaneously inhibiting their activity (Frenkel S, Singh I J, “The Effects of Fibroblast Growth Factor on Osteogenesis in the Chick Embryo,” In: FUNDAMENTALS OF BONE GROWTH: METHODOLOGY AND APPLICATIONS. Ed. AD Dixon, BG Sarnat, D. Hoyte, CRC Press, Boca Raton, Fla., USA, pp. 245-259, 1990). This effect is dose dependent, with higher and lower doses causing decreased activity and middle range doses stimulating activity (Aspenberg P, Thorngren K G, Lohmander L S, “Dose-dependent Stimulation of Bone Induction by Basic Fibroblast Growth Factor in Rats,” Acta Orthop Scand 62:481-484, 1991).

It will be appreciated that the amount of active ingredient used will vary depending upon the type of active ingredient, the specific activity of the particular active ingredient preparation employed, and the intended use of the composition. The desired amount is readily determinable by the user. For example, a composition according to the present invention may include between about 0.1% and about 4% osteoinductive substance (percent of the total reconstituted weight of the composition.

An “osteoinductive substance” refers to any substance that is capable of inducing bone formation (i.e., a material having osteogenic properties) when implanted in a body and includes demineralized bone matrix and osteoinductive factors. An “osteoinductive factor” or “bone growth factor” refers to a natural, recombinant or synthetic protein or mixture of proteins which are capable of inducing bone formation, such as the bone growth factors described in U.S. Pat. No. 5,290,763 (Poser et al.), U.S. Pat. No. 5,371,191 (Poser et al.), or U.S. Pat. No. 5,563,124 (Damien et al.), and as described in pending U.S. patent application Ser. No. 09/545,441, particularly in Example 21 (“Characterization of BP”), the disclosures of which are incorporated by reference herein in their entirety. It should be noted that while most contemplated applications of the compositions, devices and methods disclosed herein are concerned with use in humans, the same or similar products and processes work in animals as well.

Suitable osteoinductive factors may be obtained by purification of naturally occurring proteins from bone or by recombinant DNA techniques. As used herein, the term recombinantly produced osteoinductive factors refers to the production of osteoinductive factors using recombinant DNA technology. For example, nucleic acids encoding proteins having osteogenic activity can be identified by producing antibodies that bind to the proteins. The antibodies can be used to isolate, by affinity chromatography, purified populations of a particular osteogenic protein.

The amino acid sequence can be identified by sequencing the purified protein. It is possible to synthesize DNA oligonucleotides from the known amino acid sequence. The oligonucleotides can be used to screen either a genomic DNA and/or cDNA library made from, for example bovine DNA, to identify nucleic acids encoding the osteogenic protein. The correct oligonucleotide will hybridize to the appropriate cDNA thereby identifying the cDNA encoding the osteogenic protein encoding gene.

Antibodies that bind osteogenic proteins can also be used directly to screen a cDNA expression library. For example, eukaryotic cDNA sequences encoding osteogenic proteins can be ligated into bacterial expression vectors. The expression vectors can be transformed into bacteria, such as E. coli, which express the transformed expression vector and produce the osteogenic protein. The transformed bacteria can be screened for expression of the osteogenic protein by lysing the bacteria and contracting the bacteria with radioactively-labeled antibody.

Recombinant osteoinductive factor can be produced by transfecting genes identified according to the technique described above into cells using any process by which nucleic acids are inserted into cells. After transfection, the cell can produce recombinant osteoinductive factors by expression of the transfected nucleic acids and such osteoinductive factors can be recovered form the cells.

A number of naturally occurring proteins from bone or recombinant osteoinductive factors have been described in the literature and are suitable for use in the new collagen-based compositions and methods described above. Recombinantly produced osteoinductive factors have been produced by several entities. Creative Biomolecules of Hopkinton, Mass., USA produces a osteoinductive factor referred to as Osteogenic Protein 1 or OP1. Genetics Institute of Cambridge, Mass., USA produces a series of osteoinductive factors referred to as Bone Morphogenetic Proteins 1-13 (i.e., BMP 1-13), some of which are described in U.S. Pat. Nos. 5,106,748 and 5,658,882 and in PCT Publication No. WO 96/39,170. Purified osteoinductive factors have been developed by several entities. Collagen Corporation of Palo Alto, Calif., USA developed a purified protein mixture which is believed to have osteogenic activity and which is described in U.S. Pat. Nos. 4,774,228; 4,774,322; 4,810,691; and 4,843,063. Marshall Urist of the University of California developed a purified protein mixture which is believed to be osteogenic and which is described in U.S. Pat. Nos. 4,455,256; 4,619,989; 4,761,471; 4,789,732;

and 4,795,804. International Genetic Engineering, Inc. of Santa Monica, Calif., USA developed a purified protein mixture which is believed to be osteogenic and which is described in U.S. Pat. No. 4,804,744.

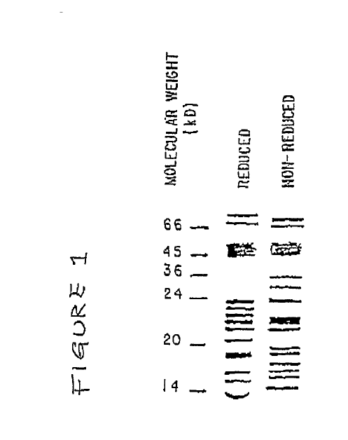

A preferred osteoinductive factor for incorporating into the new compositions, and a process for making that factor, is described in detail in U.S. Pat. No. 5,290,763. This osteoinductive factor is particularly preferred because of its high osteogenic activity and degree of purity. The osteoinductive factor of U.S. Pat. No. 5,290,763 exhibits osteoinductive activity at about 3 micrograms when deposited onto a suitable carrier and implanted subcutaneously into a rat. In one embodiment, the osteoinductive factor is an osteoinductively active mixture of proteins which exhibit the gel separation profile shown in FIG. 1 of U.S. Pat. No. 5,563,124. This gel separation profile was obtained using SDS-PAGE. The first column is a molecular weight scale which was obtained by performing SDS-PAGE on standards of known molecular weight. The second column illustrates the SDS-PAGE profile for a mixture of proteins in accordance with the present invention which have been reduced with 2-mercaptoethanol. The third column illustrates the SDS-PAGE profile for a non-reduced mixture of proteins in accordance with the present invention. Although the mixture of proteins which provide the SDS-PAGE profile illustrated therein have been found to have high osteoinductive activity, it is anticipated that mixtures of proteins having SDS-PAGE profiles which differ slightly from that illustrated therein will also be effective. For example, effective protein mixtures can include proteins that differ in molecular weight by plus or minus 5 KD from those shown therein, and can include fewer or greater numbers of proteins than those shown. Such a protein mixture may also comprise a mixture of proteins having a profile that comprises substantially all of the protein bands detected in the reduced or nonreduced SDS-PAGE profiles illustrated in that reference.

Preferred osteoinductive factors comprise an osteoinductively active mixture of proteins having, upon hydrolysis, an amino acid composition of from about 22.7 to about 26.2 mole percent acidic amino aids, about 45.0 to about 48.5 mole percent aliphatic amino acids, about 6.6 to about 8.4 mole percent aromatic amino acids and about 19.9 to about 22.8 mole percent basic amino acids. The osteoinductive factor may also have an amino acid composition of about 22.7 to about 26.2 mole percent of ASP (+ASN) and GLU (+GLN); about 45.0 to about 48.5 mole percent ALA, GLY, PRO, VAL, MET, ILE, and LEU; about 6.6 to about 8.4 mole percent TYR and PHE; and about 19.9 to about 22.8 mole percent HIS, ARG, and LYS. Another preferred osteoinductive factor is a protein mixture obtained by any of the purification processes described in U.S. Pat. No. 5,290,763 (Poser et al.).

A preferred angiogenic mixture of bone proteins is produced by a multi-step process that includes an ultrafiltration step, an anion exchange chromatography step, a cation exchange chromatography step and a high performance liquid chromatography (HPLC) purification step as described in detail below. Preferred processes for producing the angiogenic protein mixtures of the present invention are described in full detail in U.S. Pat. Nos. 5,290,763 and 5,371,191, which are incorporated herein in their entireties. The processes can be summarized as follows. In a first step, demineralized bone particles from a suitable source (such as crushed bovine bone) are subjected to protein extraction using guanidine hydrochloride. The extract solution is filtered, and subjected to a two step ultrafiltration process. In the first ultrafiltration step, an ultrafiltration membrane having a nominal molecular weight cut off (MWCO) of 100 kD is preferably employed. The retentate is discarded and the filtrate is subjected to a second ultrafiltration step using an ultrafiltration membrane preferably having a nominal MWCO of about 10 kD. The retentate is then subjected to diafiltration to substitute urea for guanidine. The protein-containing urea solution is then subjected to sequential ion exchange chromatography, first anion exchange chromatography followed by cation exchange chromatography. For the anion exchange process, a strongly cationic resin is used, preferably having quaternary amine functional groups. Typically, the eluant for the anion exchange process has a conductivity from about 10,260 micromhos (μmhos) (1.026×10<−2> siemens (S)) to about 11,200 μmhos (1.120×10<31 2>S). For the cation exchange process, a strongly anionic resin is used, preferably having sulfonic acid functional groups. The eluant for the cation exchange process typically has a conductivity from about 39,100 μmhos (3.91×10<−2>S) to about 82,700 μmhos (8.27×10<−2>S) or more.

In the process described above, the proteins are advantageously kept in solution. According to the present invention, the proteins produced by the above process are then subjected to HPLC. The HPLC process preferably utilizes a column containing hydrocarbon-modified silica packing material. The proteins can be loaded onto the HPLC column in a solution of aqueous trifluoracetic acid or other suitable solvent, such as heptafluorobutyric acid, hydrochloric or phosphoric acid. Preferably, a trifluoracetic acid solution having a concentration of from about 0.05 percent by volume to about 0.15 percent by volume, and more preferably about 0.1 percent by volume trifluoracetic acid is used.

Proteins are eluted from the HPLC column with an organic solvent/water mixture suitable for obtaining the desired proteins. A preferred eluant in the HPLC process is an acetonitrile solution. The preferred eluant typically has an acetonitrile concentration which varies, during elution, from about 30 percent by volume to about 45 percent by volume. In preferred embodiments, the acetonitrile concentration in the eluant is increased in increments of between about 0.30 percent by volume and about 0.40 percent by volume per minute until the desired highest concentration of acetonitrile is achieved. Proteins can be recovered from the HPLC process eluant by means generally known in the art. A preferred angiogenic fraction of the eluted proteins occurs when the acetonitrile concentration in the eluant is between about 33 percent by volume and about 37 percent by volume.

The purification processes described above yield novel angiogenic protein mixtures. Because they comprise mixtures of proteins, these angiogenic factors are most easily described in terms of their properties. Hence, in one embodiment of the present angiogenic factor, the factor is a mixture of a number of proteins having the sodium dodecyl sulfate polyacrylamide gel electrophoresis (SDS-PAGE) profile shown in FIG. 1.

Another characterization of the present invention is a mixture of proteins having a preferred amino acid composition of about 20-25 mole percent of acidic amino acids [ASP(+ASN) and GLU(+GLN)]; about 10-15 mole percent of hydroxy amino acids (SER and THR); about 35-45 mole percent aliphatic amino acids (ALA, GLY, PRO, MET, VAL, ILE, and LEU); about 4-10 mole percent aromatic amino acids (TYR and PHE); and about 10-20 mole percent basic amino acids (HIS, ARG and LYS). More particularly, this embodiment of the angiogenic protein mixture amino preferably has an amino acid composition of about 23.4 mole percent of acidic amino acids [ASP(+ASN) and GLU(+GLN)]; about 13.5 mole percent of hydroxy amino acids (SER and THR); about 40.0 mole percent aliphatic amino acids (ALA, GLY, PRO, MET, VAL, ILE, and LEU); about 6.8 mole percent aromatic amino acids (TYR and PHE); and about 16.6 mole percent basic amino acids (HIS, ARG and LYS). (TRP, CYS and 1/2 CYS were not measured and are not included in the calculation of mole percent.)

An alternative embodiment of the present angiogenic factor can be defined as a different fraction of the total protein stream exiting the HPLC process. More particularly, the proteins eluted when the eluant has an acetonitrile concentration of from about 37 to about 39.5 percent by volume have been found to have surprising angiogenic activity. The mixture defined in this manner contains hundreds of natural proteins. It is believed that the angiogenic activity of proteins obtained in this manner may be further enhanced by selecting smaller fractions of the eluant and quantitatively comparing the angiogenic activity of each fraction.

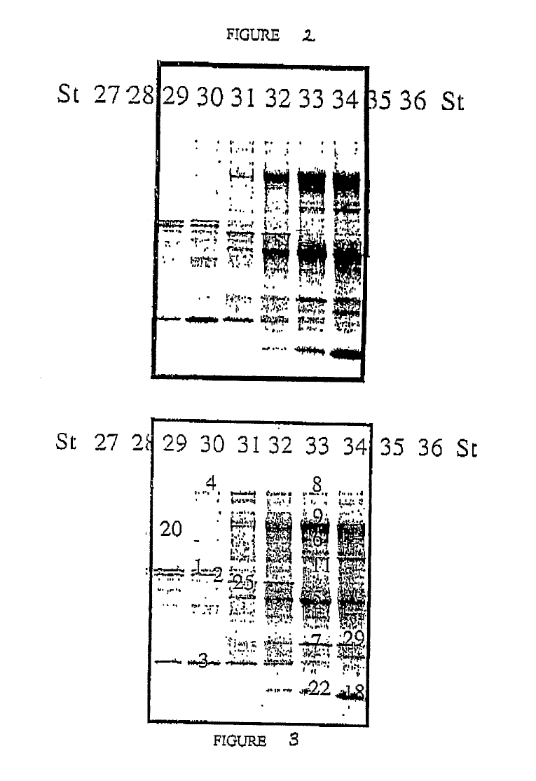

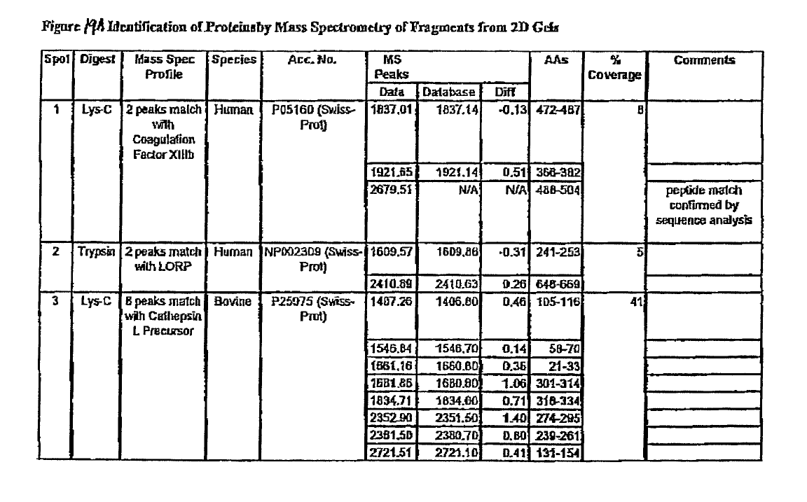

In addition to the foregoing, BP has been partially characterized as follows: high performance liquid chromatography (HPLC) fractions have been denatured, reduced the DTT, and separated by sodium dodecyl sulfate polyacrylamide gel electrophoresis (SDS-PAGE). One minute HPLC fractions from 27 to 36 minutes are shown in FIG. 2. Size standards (ST) of 14, 21, 31, 45, 68 and 97 kDa were obtained as Low range size standards from BIORAD™ and are shown at either end of the coomassie blue stained gel. In the usual protocol, HPLC fractions 29through 34 are pooled to produce BP (see boxes, FIGS. 2 and 3), as shown in a similarly prepared SDS-PAGE gel in FIG. 17B.

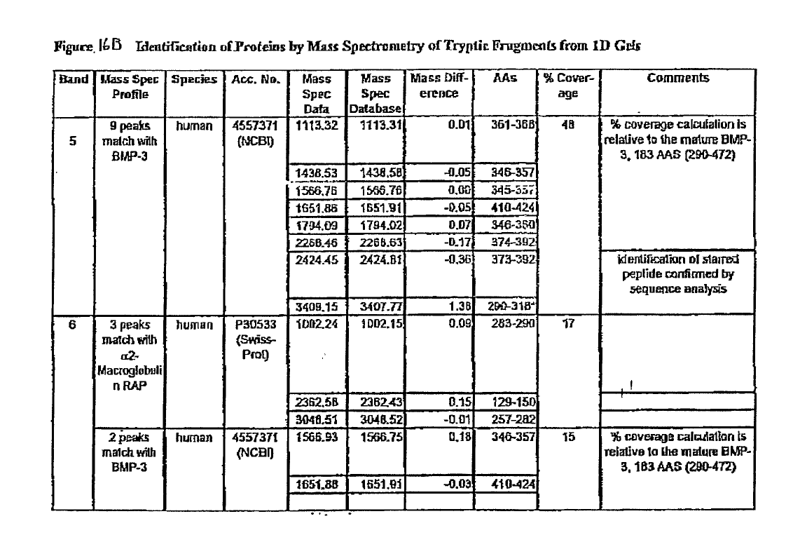

The various components of BP were characterized by mass spectrometry and amino acid sequencing of tryptic fragments where there were sufficient levels of protein for analysis. The major bands in the ID gel (as numerically identified in FIG. 3) were excised, eluted, subjected to tryptic digestion and the fragments were HPLC purified and sequenced. The sequence data was compared against known sequences, and the best matches are shown in FIGS. 12A-B. These identifications are somewhat tentative, in that only portions of the entire proteins have been sequenced and, in some cases, there is variation between the human and bovine analogs for a given protein.

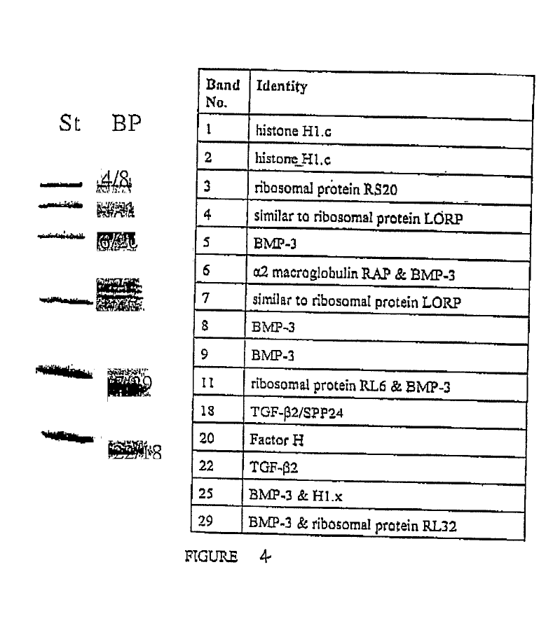

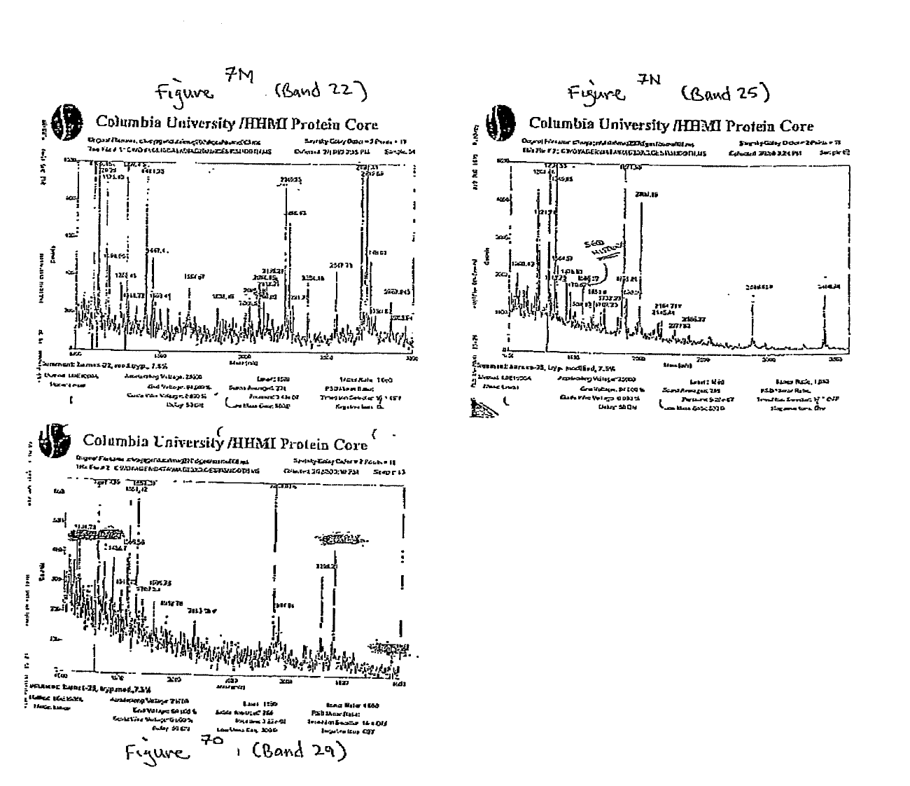

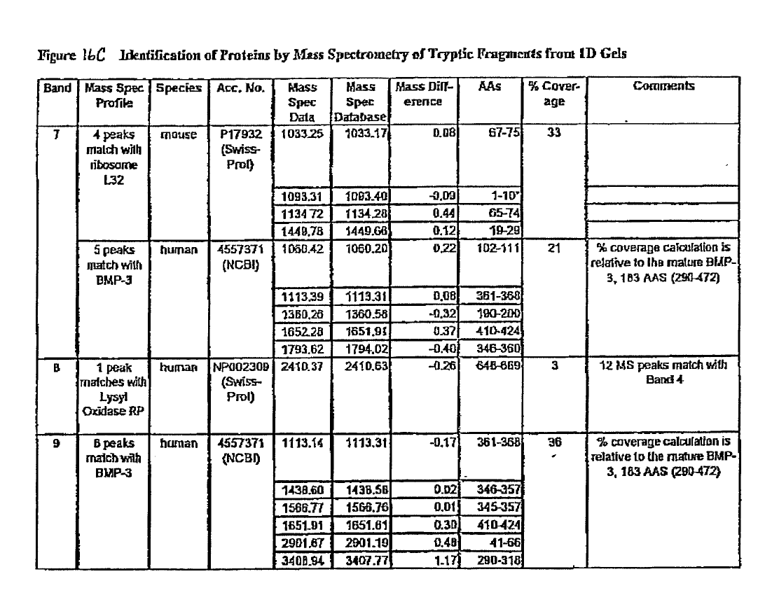

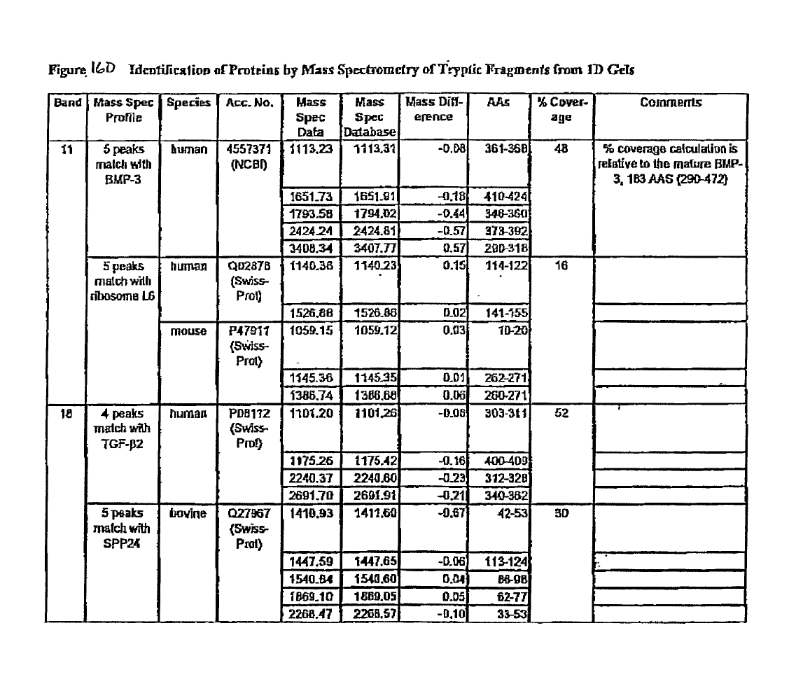

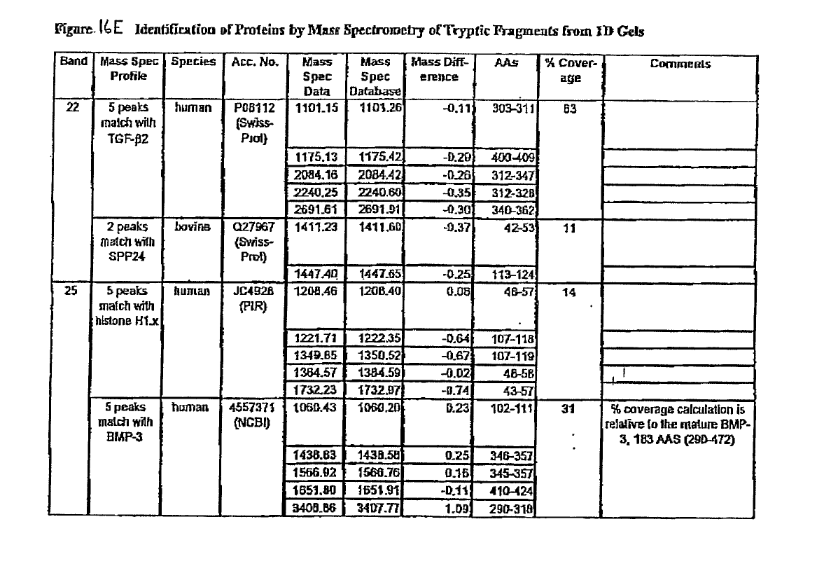

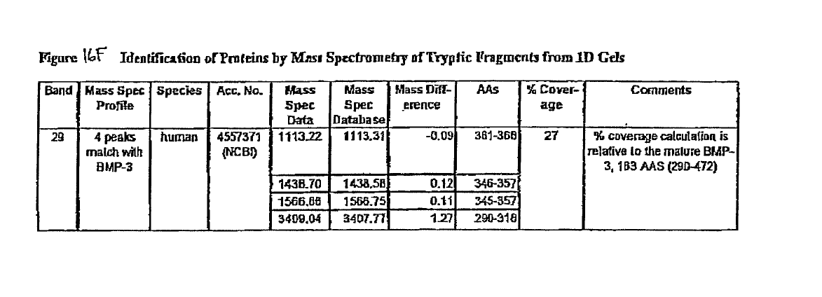

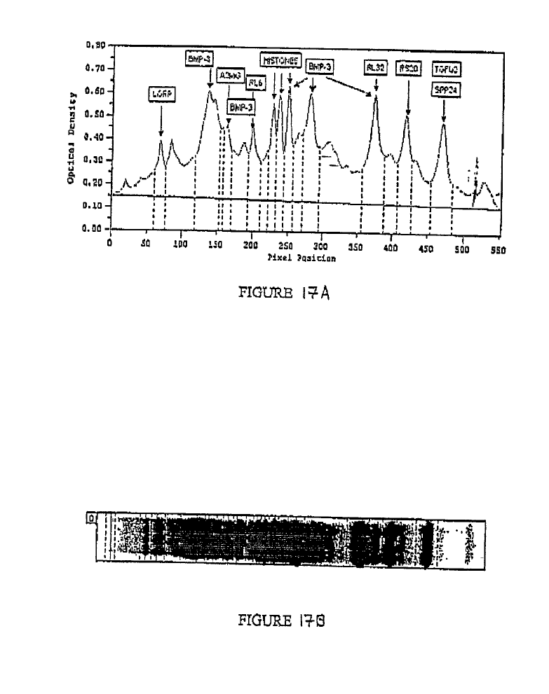

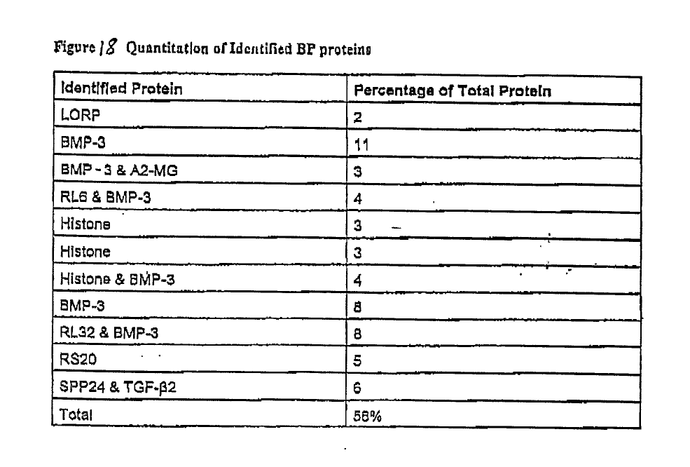

The same tryptic protein fragments were analyzed by mass spectrometry and the mass spectrograms are shown in FIGS. 7A-O. The tabulated results and homologies are shown in FIGS. 16A-F, which provide identification information for the bands identified in FIGS. 3-4. As above, assignment of spot identity may be tentative based on species differences and post translational modifications. A summary of all protein identifications for 1 d gels is shown in FIG. 4.

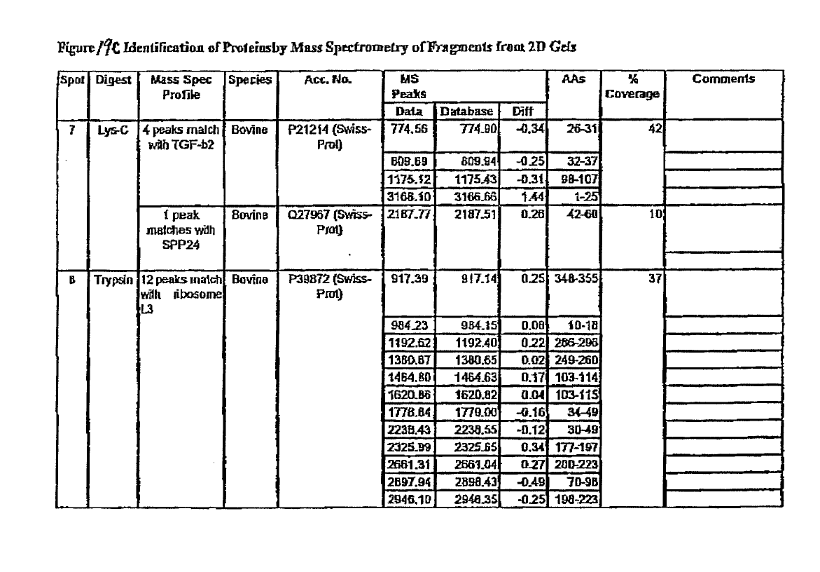

The identified protein components of BP, as described in FIGS. 15A-B, 16A-F and 19A-D, were quantified as shown in FIGS. 17A and 17B. FIG. 17B is a stained SDS-PAGE gel of BP and FIG. 17A represents a scanning densitometer trace of the same gel. The identified proteins were labeled and quantified by measuring the area under the curve. These results are presented in FIG. 18 as a percentage of the total peak area.

Thus, there are 11 major bands in the BP SDS-PAGE gel, representing about 60% of the protein in BP. The identified proteins fall roughly into three categories: the ribosomal proteins, the histones, and growth factors, including bone morphogenic factors (BMPs). It is expected that he ribosomal proteins may be removed from the BP without loss of activity, since these proteins are known to have no growth factor activity. Upon this separation, the specific activity is expected to increase correspondingly.

It is expected that the histone and ribosomal proteins may be removed from the BP with no resulting loss, or even with an increase, in specific activity. It is expected that histones can removed from the BP cocktail by immunoaffinity chromatography, using either specific histone protein antibodies or a pan-histone antibody. The histone depleted BP (BP-H) produced in this manner may be suitable for wound healing. Similarly, the mixture produced when the known ribosomal proteins are stripped from the BP cocktail (BP-R) may be suitable for wound healing.

An SDS-PAGE gel of BP was also analyzed by Western immunoblot with a series of antibodies, as listed in FIG. 14. Visualization of antibody reactivity was by horseradish peroxidase conjugated to a second antibody and using a chemiluminescent substrate. Further, TGF-β1 was quantified using commercially pure TGF-β1 as a standard and was determined to represent less than 1% of the BP protein The antibody analysis indicated that each of the proteins listed in FIG. 14 is present in BP.

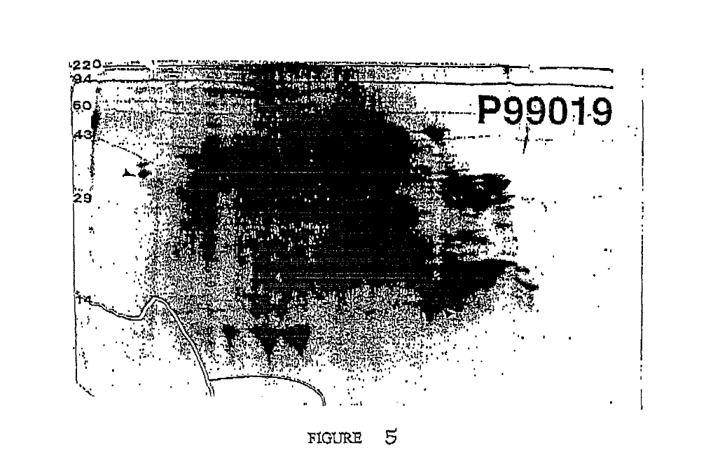

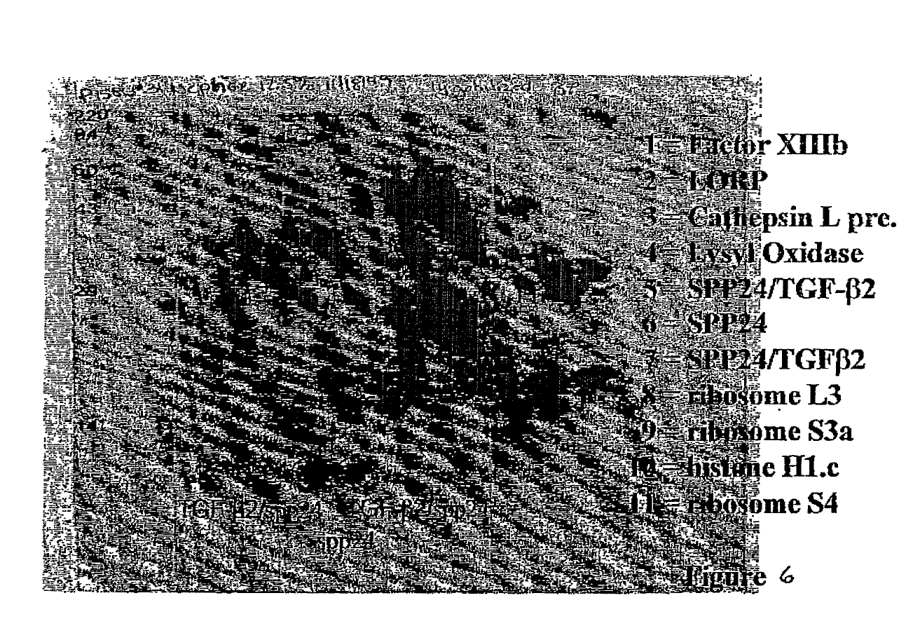

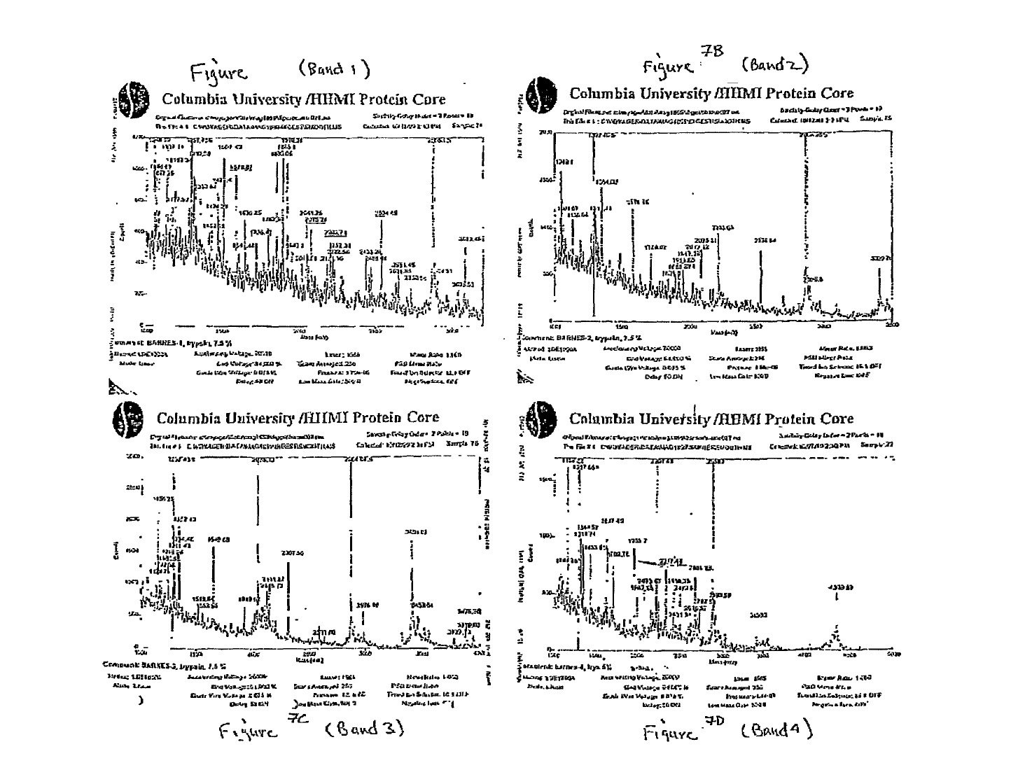

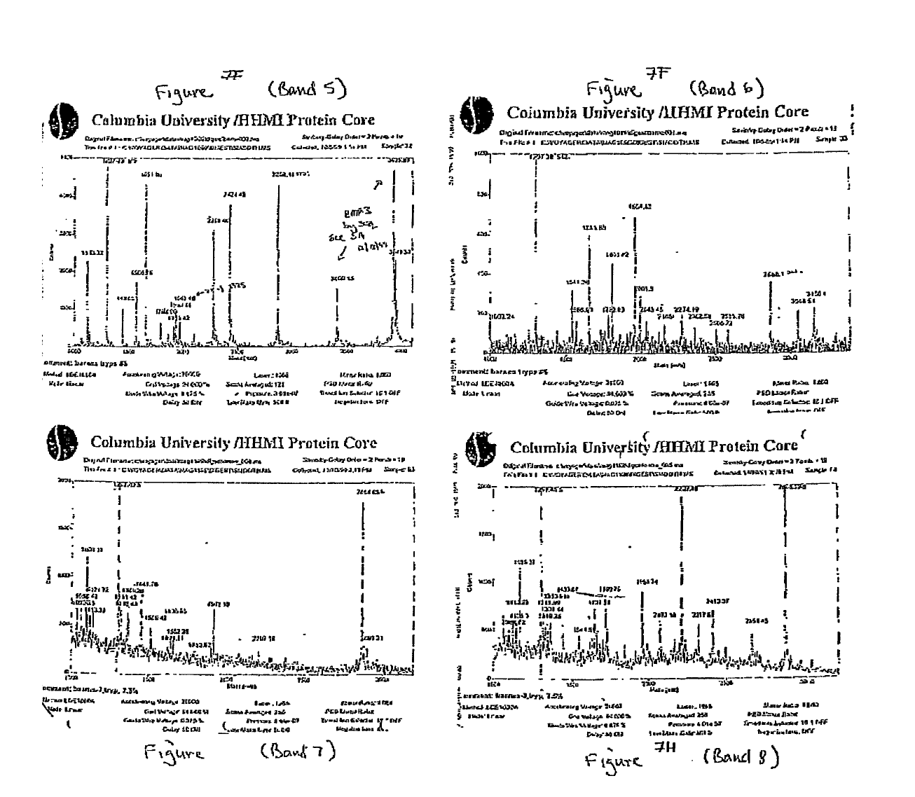

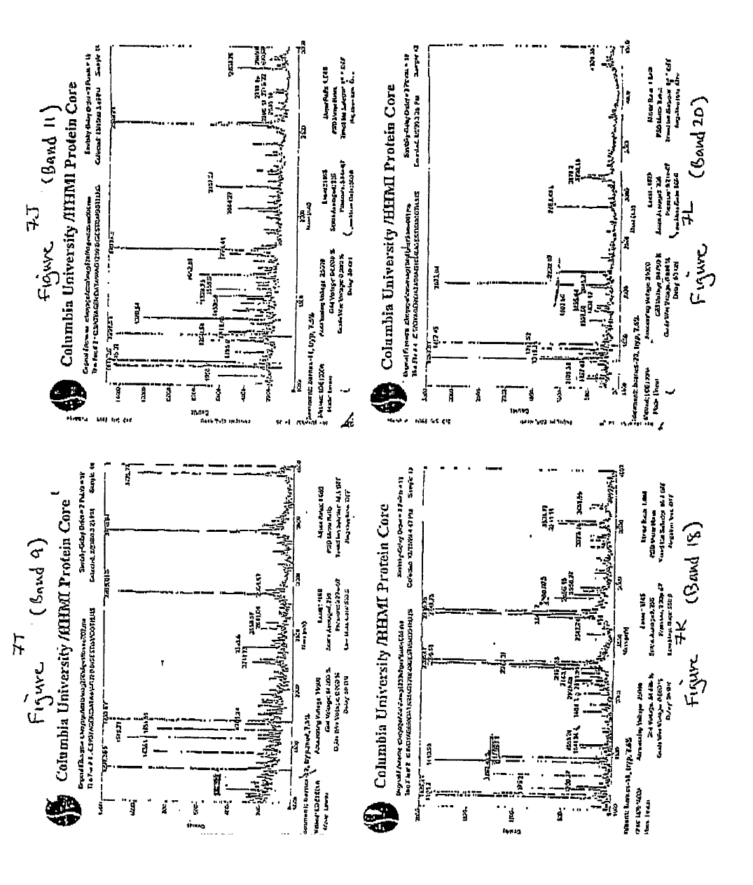

The BP was further characterized by 2-D gel electrophoresis, as shown in FIGS. 5-6. The proteins are separated in horizontal direction according to charge (pI) and in the vertical direction by size as described in two-dimensional electrophoresis adapted for resolution of basic proteins was performed according to the method of O'Farrell et al. (O'Farrell, P. Z., Goodman, H. M. and O'Farrell, P. H., Cell, 12: 1133-1142, 1977) by the Kendrick Laboratory (Madison, Wis.). Two-dimensional gel electrophoresis techniques are known to those of skill in the art. Non-equilibrium pH gradient electrophoresis (“NEPHGE”) using 1.5% pH 3.5-10, and 0.25% pH 9-11ampholines (Amersham Pharmacia Biotech, Piscataway, N.J.) was carried out at 200 V for 12 hrs. Purified tropomyosin (lower spot, 33,000 KDa, pI 5.2), and purified lysozyme (14,000 KDa, pI 10.5-11) (Merck Index) were added to the samples as internal pI markers and are marked with arrows.

After equilibration for 10 min in buffer “0” (10% glycerol, 50 mM dithiothreitol, 2.3% SDS and 0.0625 M tris, pH 6.8) the tube gel was sealed to the top of a stacking gel which is on top of a 12.5% acrylamide slab gel (0.75 mm thick). SDS slab gel electrophoresis was carried out for about 4 hrs at 12.5 mA/gel.

After slab gel electrophoresis two of the gels were coomassie blue stained and the other two were transferred to transfer buffer (12.5 mM Tris, pH 8.8, 86 mM Glycine, 10% MeoH) transblotted onto PVDF paper overnight at 200 mA and approximately 100 volts/two gels. The following proteins (Sigma Chemical Co., St. Louis, Mo.) were added as molecular weight standards to the agarose which sealed the tube gel to the slab gel: myosin (220,000 KDa), phosphorylase A (94,000 KDa), catalase (60,000 KDa), actin (43,000 KDa), carbonic anhydrase (29,000 KDa) and lysozyme (14,000 KDa). FIG. 5 shows the stained 2-D gel with size standards indicated on the left. Tropomyosin (left arrow) and lysozyme (right arrow) are also indicated.

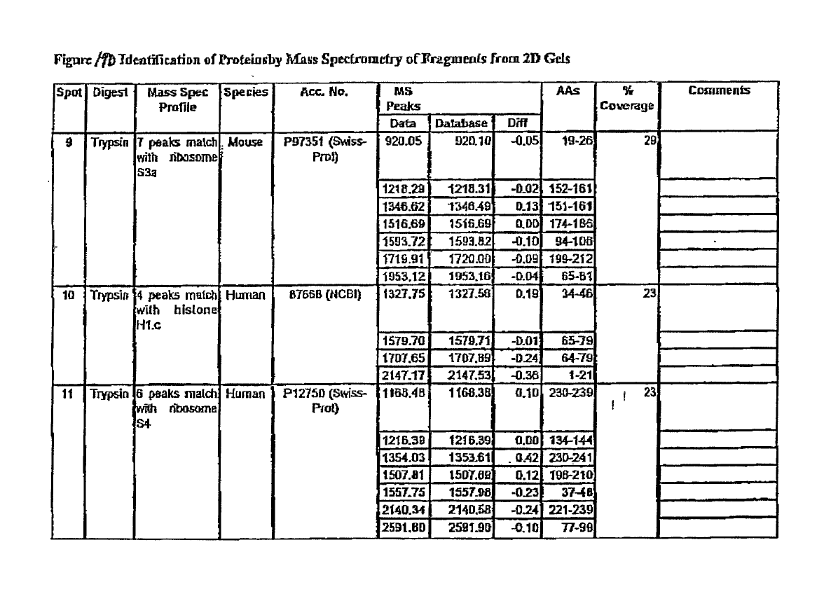

The same gel is shown in FIG. 6 with several identified proteins indicated by numbered circles. The proteins were identified by mass spectrometry and amino acid sequencing of tryptic peptides, as described above. The identity of each of the labeled circles is provided in the legend of FIG. 6 and the data identifying the various protein spots is presented in FIGS. 19A-D.

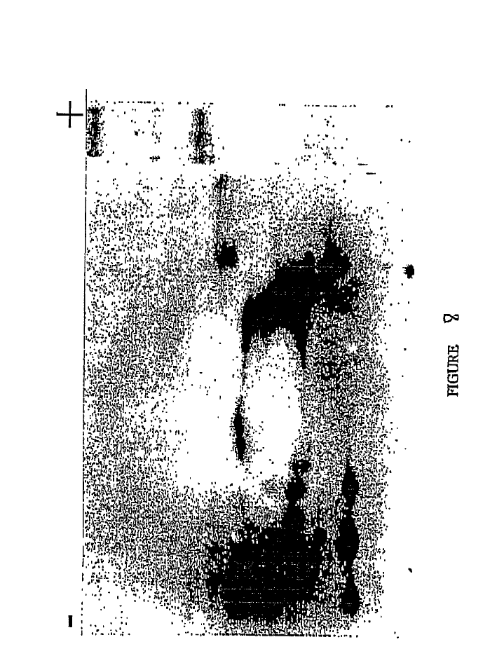

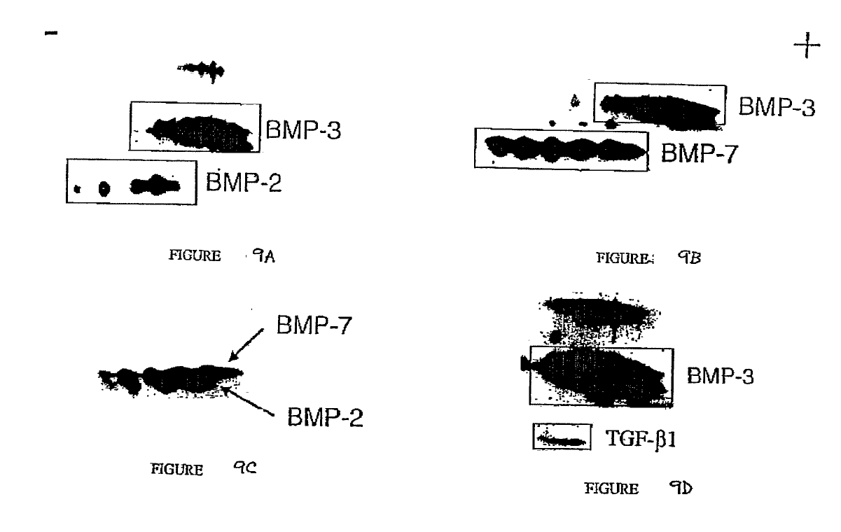

Because several of the proteins migrated at more than one size (e.g., BMP-3 migrating as 6 bands) investigations were undertaken to investigate the extent of post-translation modification of the BP components. Phosphorylation was measured by anti-phosphotyrosine immunoblot and by phosphatase studies. FIG. 8 shows a 2-D gel, electroblotted onto filter paper and probed with a phosphotyrosine mouse monoclonal antibody by SIGMA (# A-5964). Several proteins were thus shown to be phosphorylated at one or more tyrosine residues.

Similar 2-D electroblots were probed with BP component specific antibodies, as shown in FIGS. 9A-D. The filters were probed with BMP-2, BMP-3 (FIG. 9A), BMP-3, BMP-7 (FIG. 9B), BMP-7, BMP-2 (FIG. 9C), and BMP-3 and TGF-β1 (FIG. 9D). Each shows the characteristic, single-size band migrating at varying pI, as is typical of a protein existing in various phosphorylation states.

For the phosphatase studies, BP in 10 mM HCl was incubated overnight at 37° C. with 0.4units of acid phosphatase (AcP). Treated and untreated samples were added to lyophilized discs of type I collagen and evaluated side by side in the subcutaneous implant rat bioassay, as previously described in U.S. Pat. Nos. 5,290,763, 5,563,124 and 5,371,191. Briefly, 10 (g of BP in solution was added to lyophilized collagen discs and the discs implanted subcutaneously in the chest of a rat. The discs were then recovered from the rat at 2 weeks for the alkaline phosphotase (“ALP”—a marker for bone and cartilage producing cells) assay or at 3 weeks for histological analysis. For ALP analysis of the samples, the explants were homogenized and levels of ALP activity measured using a commercial kit. For histology, thin sections of the explant were cut with a microtome, and the sections stained and analyzed for bone and cartilage formation.

Both native- and phosphatase-treated BP samples were assayed for morphogenic activity by mass of the subcutaneous implant (explant mass) and ALP score. The results showed that AcP treatment reduced the explant mass and ALP score from 100% to about 60%. Thus, phosphorylation is important for BP activity.

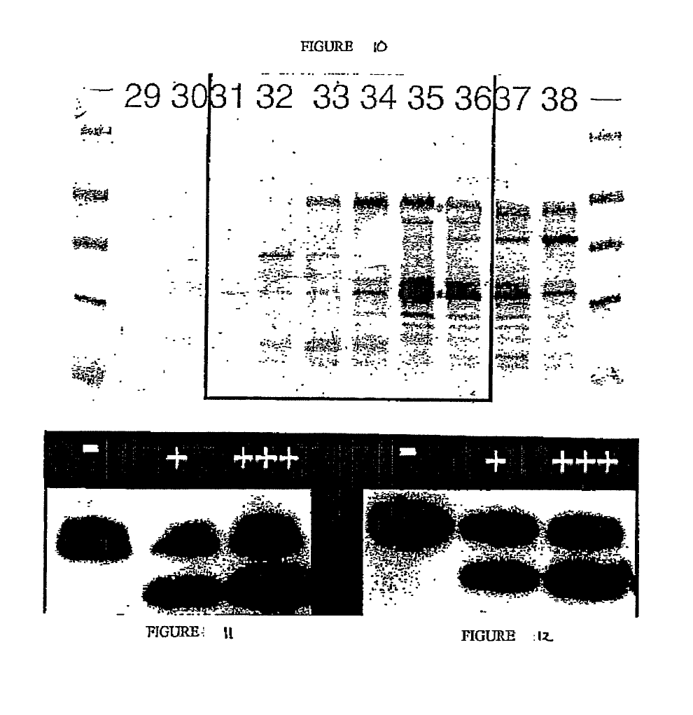

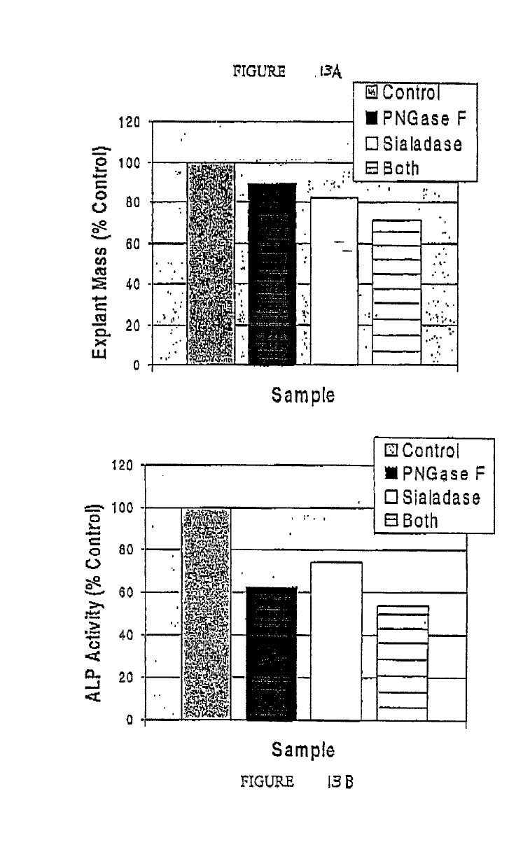

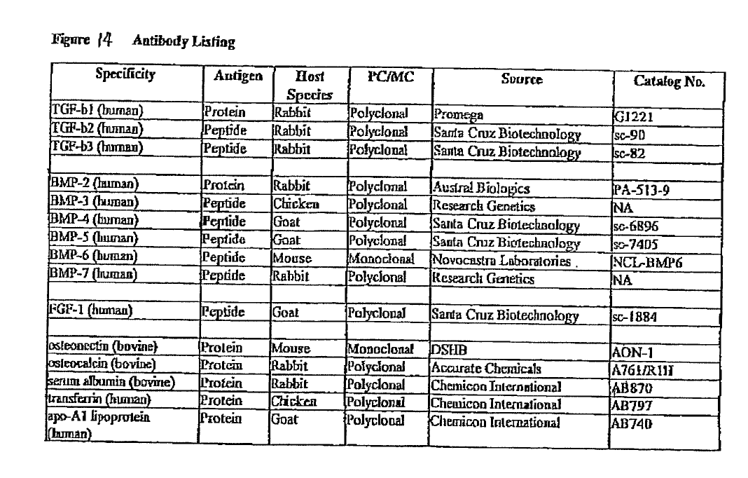

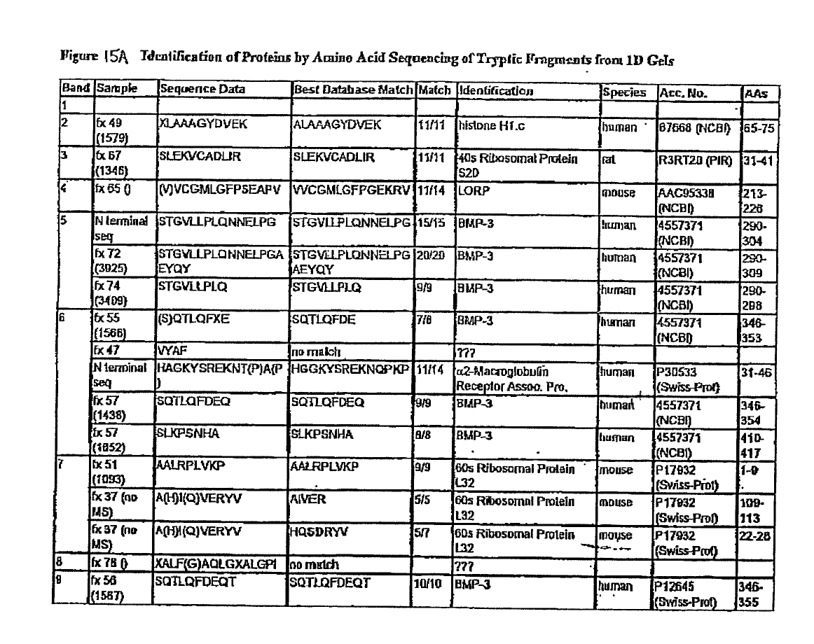

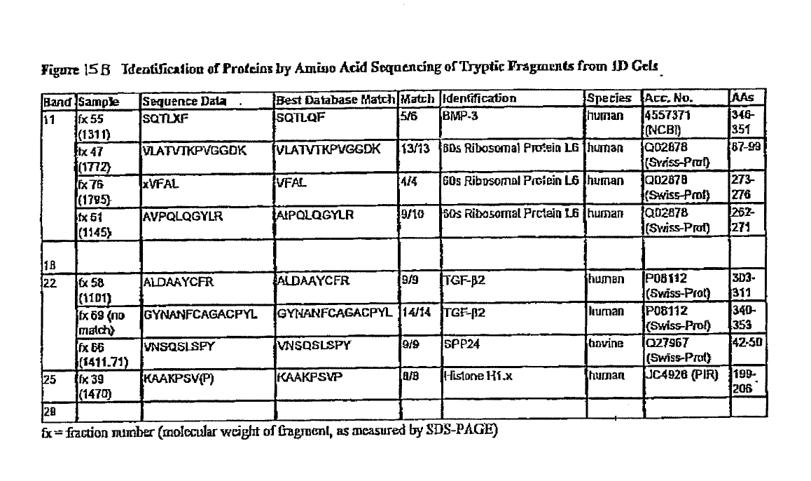

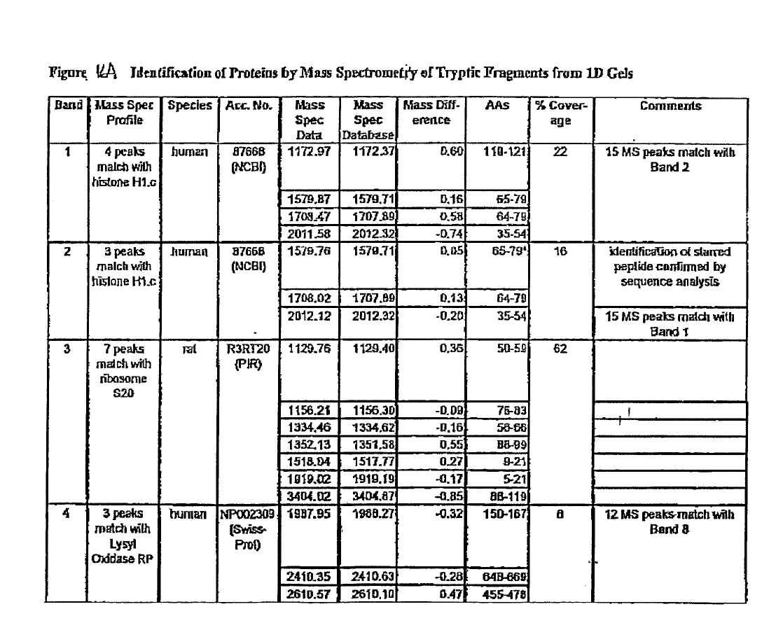

The BP was also analyzed for glycosylation. FIG. 10 shows an SDS-PAGE gel stained with periodic acid schiff (PAS)—a non-specific carbohydrate stain, indicating that several of the BP components are glycosylated (starred protein identified as BMP-3). FIGS. 11-12 show immunodetection of two specific proteins (BMP-7, FIG. 14 and BMP-2, FIG. 15) treated with increasing levels of PNGase F (Peptide-N-Glycosidase F). Both BMP-2 and BMP-7 show some degree of glycoslyation in BP, but appear to have some level of protein resistant to PNGase F as well (plus signs indicate increasing levels of enzyme). Functional activity of PNGase F and sialadase treated samples were assayed by explant mass and by ALP score, as shown in FIGS. 13A and 13B, which shows that glycosylation is required for full activity.

In summary, BMPs 2, 3 and 7 are modified by phosphorylation and glycosylation. These post-translation modifications affect protein morphogenic activity, 33% and 50% respectively, and care must be taken in preparing BP not to degrade these functional derivatives.

The compositions and methods disclosed and claimed herein can be made and executed without undue experimentation in light of the present disclosure. While the compositions and methods of this invention have been described in terms of preferred embodiments, it will be apparent to those of skill in the art that variations may be applied to the method and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Demineralized Bone Matrix

Demineralized bone matrix (DBM) suitable for use as a bulking material in some of the new compositions can be prepared in a particulate form as described by Glowacki et al. in U.S. Pat. No. 4,440,750. Alternatively, the demineralized bone material can be prepared by grinding a bone, demineralizing it with 0.6-1.2 M HCl solution, washing with a phosphate buffered solution, washing with ethanol and drying it. Preferably the average particle size is about 125-850μm. Satisfactory demineralized bone material can also be obtained from a commercial bone or tissue bank, for example, from AlloSource (Denver, Colo.) or Osteotech (Eatontown, N.J.). Although generally less preferred than DBM, calcium carbonate and other calcium salts may also be used as a bulking material.

Assessment of Osteogenic Activity

Induction of bone formation can be determined by a histological evaluation showing the de novo formation of bone with accompanying osteoblasts, osteoclasts, and osteoid matrix. For example, osteoinductive activity of an osteoinductive factor can be demonstrated by a test using a substrate onto which material to be tested is deposited. A substrate with deposited material is implanted subcutaneously in a test animal. The implant is subsequently removed and examined microscopically for the presence of bone formation including the presence of osteoblasts, osteoclasts, and osteoid matrix. A suitable procedure for assessing osteoinductive activity is illustrated in Example 5 of U.S. Pat. No. 5,290,763. Although there is no generally accepted scale of evaluating the degree of osteogenic activity, certain factors are widely recognized as indicating bone formation. Such factors are referenced in the scale of 0-8 which is provided in Table 3 of example 1 of U.S. Pat. No. 5,563,124. The 0-4 portion of this scale corresponds to the scoring system described in U.S. Pat. No. 5,290,763, which is limited to scores of 0-4. The remaining portion of the scale, scores 5-8, references additional levels of maturation of bone formation. The expanded scale also includes consideration of resorption of collagen, a factor which is not described in U.S. Pat. No. 5,290,763.

Representative new osteogenic compositions and devices employing such compositions, their manner of making and their use in dental procedures such as repairing periodontal defects or in spinal fusion applications, are described in the following

### EXAMPLES

### Example 1

### Composition for Periodontal Bone Regeneration

Purified fibrillar bovine tendon type I collagen is dispersed in a slightly acidic aqueous solution, preferably about 10 mm HCl, to provide a concentration of about 1-3% (wt./vol.), preferably 1-2%. An effective amount of an active ingredient or agent, preferably an osteoinductive substance such as a bone growth factor, is also combined in the dispersion. The amount of agent added depends on the specific activity and purity of the agent. An “effective amount” of a biologically active agent is an amount sufficient to elicit a desired biological response. Suitable osteoinductive substances and other active agents are described above under “General Materials and Methods.” In the case of a purified bone growth factor, the effective amount is preferably between about 0.1% (by weight of the total weight of the final composition) and about 3-4%. One suitable bone growth factor is SBI Growth Factor Mixture GFm, as described in U.S. Pat. No. 5,290,763. This and other suitable active agents and osteoinductive substances or factors are discussed in more detail above in General Materials and Methods, in the subsection entitled “Osteoinductive Substances and Other Active Agents.” For convenience, the combination of collagen, osteogenic substance, other active agent, if any, and the dilute acid solution will be referred to in the present examples as the “collagen-growth factor mixture.”

A suitable amount of collagen is placed in the barrel of a sterile syringe and the syringe plunger is then reinserted into the barrel to retain the dry material near the outlet end of the syringe.

A sufficient amount of dilute acid in water, preferably about 10 mM HCl containing the desired amount of growth factor, is introduced into a second sterile syringe, taking care to expel any trapped air from the syringe. Preferably the amounts of water and collagen are sufficient to yield an approximately 1-4% (wt./vol.) collagen dispersion, preferably about 1-2% for most periodontal applications.

The outlet ends of the two syringes are then connected together, preferably using a standard Luer type adapter, or other similar connector. Any excess air is removed and the syringes recoupled. The second syringe plunger is then depressed to force the liquid into the first syringe, which contains the dry material, and rehydration of the collagen-growth factor mixture commences. Alternatively, the liquid and dry ingredients may be initially placed in the same syringe, although this is not presently preferred. The first syringe plunger automatically retracts as the second, opposite, plunger is depressed. The plunger of the second syringe is then depressed to force the liquid and the at least partially rehydrated collagen-growth factor mixture through the syringe nozzles and connector and into the first syringe. This exchange of ingredients back and forth between the two syringes is repeated for a total of about 5 passes, after which the dispersion is allowed to rest in the syringe for at least about 30 minutes, preferably about 45-60 min., more preferably 60 min. The liquid can be observed to slowly thicken. The somewhat thickened dispersion is then expressed back and forth between the syringes about 5 more times, after which it is again allowed to rest in the syringe for a longer period of time, preferably overnight. Further gradual thickening is apparent during that interval. The mixing step is preferably carried out at room temperature while final incubation is carried at refrigerated temperature, preferably about 2-8° C. Without wishing to be bound by a particular theory, the inventor believes that shear forces and/or other hydrodynamic forces applied to the collagen as it passes into and through the reduced diameter portion of the syringe assembly aligns or orients the collagen fibers and/or facilitates natural crosslinking.

The thick, gel-like dispersion is then extruded from the syringe into one or more molds, frozen at about −70° C., and then lyophilized to dryness. This molded product, produced by the above-described syringe exchange process, is less porous than a counterpart product produced by simply rehydrating and stirring the collagen and bone growth factor in 10 mM HCl and, when rehydrated, provides distinct handling differences.

Although other types of collagen may be processed similarly, use of bovine type I tendon collagen is highly preferred in this method. In preliminary studies, it was observed that other types of collagen, when combined with a growth factors, demonstrated less osteogenic activity than bovine type I tendon collagen together with the same growth factors.

The product may be sterilized by dialysis, irradiation (e.g. using g-radiation), filtration, chemical treatment (e.g., using ethylene oxide), or other known sterilization methods, as appropriate. Preferably, the composition is lyophilized to a dry solid before being sterilized. When sterilizing the material using a chemical treatment, it is preferred that the material be lyophilized to a dry solid prior to be sterilized. Lyophilization removes water and prevents any chemical reaction which may occur between the chemical used for sterilization (e.g., ethylene oxide) and water. Alternatively, the composition can be made in an aseptic environment, thereby eliminating the need for a separate sterilization step.

For ease of production in a small-scale process, it is preferable to use the above-described two syringe multiple pass technique for producing a collagen-bone growth factor dispersion with the desired physical characteristics, and, ultimately, products having the desired porosity, resilience and tensile strength characteristics. Especially in larger scale commercial production, however, an alternative method of dispersing the collagen-growth factor mixture may be preferred, provided that any alternative apparatus creates physical forces on the collagen mixture (e.g., shear forces and/or alignment of collagen fibers) substantially equivalent to those created by the two syringe multiple pass technique described in the present examples.

The lyophylized collagen-growth factor product is preferably packaged for subsequent periodontal implantation by placing the molded, dehydrated composition in a suitable protective covering and sterilizing it (e.g., by treatment with ethylene oxide). A preferred sterile packaging system includes a small plastic bowl or tray assembly containing the dry collagen-growth factor matrix material covered by a removable lid such as an adherent TYVEK™ seal. The bowl is of appropriate size for adding a liquid to the dry matrix material and mixing in a bulking material to produce the final product: a readily shapeable, implantable paste or putty that can stay in place after implantation. If desired, a suitable amount of a bulking material, such as particulate demineralized bone matrix, may be included as part of a kit, as discussed in more detail in Example 3, below.

In a surgical setting, the health care practitioner opens the sterile packaging and rehydrates the dried collagen-growth factor matrix with sterile water for injection. The wetted matrix initially has some degree of resilience, but when more fully rehydrated falls apart and is readily mixed with another matrix or bulking material. The surgeon breaks or teases the rehydrated matrix apart and mixes in a predetermined amount of a suitable bulking material. For example, a single unit of molded matrix, suitable for implanting in a dental socket, might weigh about 7-9 mg. To this amount of dry product, about 100 μl water for injection and about 40 mg DBM may be added, for example. Alternatively, the amount of water and bulking material mixed into the rehydrated matrix material may be empirically determined by the user, depending upon the consistency desired for the particular application or that is preferred by the user. For instance, just enough water is added to soften the dried collagen-growth factor matrix enough for it to come apart with gentle probing, and just enough powdered DBM is stirred into the wet matrix material to achieve a paste-like consistency that the surgeon can shape and implant. The preferred bulking material is particulate demineralized bone matrix (DBM), which is prepared as described in more detail under “General Materials and Methods,” or can be obtained from known commercial sources. The final product, a workable osteogenic putty or paste-like material (which can be less cohesive than the putty-like material described in U.S. patent application Ser. No. 09/023,617) is then shaped by the surgeon into the desired configuration, which may be structurally similar to a wad of cotton, and the shaped device is then implanted at the site where bone growth is desired and molded to fit the particular defect area. For instance, the shaped device might be placed into a dental socket.

A composition prepared as described above and containing bone growth factor was evaluated for osteogenic activity in a clinical furcation defect modal. Osteogenic bone formation was evaluated by measuring horizontal and vertical attachment levels. Results indicated bone formation that resulted in HAL and VAL augmentation of 2-3½ times that obtained with a placebo.

### Example 2

### Composition for Use with a Spinal Cage for Spinal Fusion

In spinal fusion operations in which it is desired to substantially immobilize two vertebrae with respect to each other, titanium cages or similar implantable devices may be are placed in the space between two vertebral bodies. An osteogenic material is packed into and around the cages to obtain bone formation through and around the cages, thus fusing together two vertebrae and stabilizing the spine.

The present invention provides a new composition that is suitable for insertion into a such a spinal cage, the composition being prepared similarly to that described above for preparing a periodontal composition or device. In this case, however, the collagen concentration is preferably greater to provide a stronger, more resilient or sponge-like product, the number of passages through the syringe assembly is increased, and the bulking material is preferably omitted. Purified fibrillar bovine tendon type I collagen is dispersed in an acidic aqueous solution, preferably about 10 mm HCl, to provide a concentration of about 2-4% (wt./vol.) collagen. An osteoinductive substance such as a bone growth factor is included in the mixture. If desired, another active ingredient or agent may also be included. As in Example 1, the amount of added osteoinductive substance or other active agent depends on the type, specific activity and purity of the additive, as well as the species receiving the implant. The desired amount of collagen, growth factor and dilute acid in water is passed back and forth in the coupled syringes as described in Example 1, except the first set of passes consists of about 100 passes. The mixture is allowed to stand for about an hour, and is then subjected to a second set of about 100-150 passes, or more, after which the mixture is allowed to stand in a syringe overnight. Preferably the total number of passes are divided about equally between the first and second set of passes. The resulting collagen-bone growth factor mixture is thicker than that obtained in Example 1. This dispersion, which is quite thick yet still extrudable, is expressed from the syringe into one or more molds, frozen at about 70° C., and then lyophilized to dryness. The shape is dependent on the choice of implant and is designed to interlock inside the cage, or other device, with a portion preferably directly opposed to the bony surfaces of the vertebral bodies. Also, additional cross-linking after molding and lyophilization can be performed, using, for example, formaldehyde vapor, dehydrothermal crosslinking, or other crosslinking method if additional strengthening of the product is desired for particular applications.

The lyophilized collagen-growth factor product intended for use with a spinal cage is packaged by placing the dehydrated, molded composition in a suitable protective covering and sterilizing it by treatment with ethylene dioxide, for example. A preferred sterile packaging system includes a bowl or tray assembly containing the dry collagen-growth factor matrix material covered with a removable lid such as an adherent TYVEK™ seal. The bowl or tray is of appropriate size for adding a liquid to the matrix material to produce the final rehydrated product: a resilient, conformable sponge-like material that holds its shape yet is deformable. It can be easily handled, compressed and inserted into a spinal cage by the surgeon. After implantation, as the composition becomes more fully hydrated, it will expand to fill the cage.

In a surgical setting, the health care practitioner opens the sterile packaging and rehydrates the dried growth factor-containing collagen matrix with preferably sterile water, or less preferably, isotonic saline. This wetted collagen-growth factor matrix is more resilient than the counterpart product of Example 1, and does not fall apart after wetting. Due to the strengthening of the matrix as a result of the additional passes in the syringe assembly, the surgeon can conveniently insert the rehydrated sponge directly into the cage without needing to first add a bulking material, such as DBM. The rehydrated product is then placed in a metal cage fusion device, or spinal cage, for implantation in the intervertebral space of a subject. Such spinal cages are well known in the art. One such device is disclosed in U.S. Pat. No. 5,397,364 to Kozak et al. The wetted matrix swells in situ after placement in the cage and substantially conforms to the shape of the cage.

As an alternative to placing the matrix inside a spinal cage, one of the new collagen-based compositions or devices may also be used in spinal fusion operations by placing the composition or device between adjacent spinous and transverse processes so that upon bone formation throughout the material, two adjacent vertebrae are joined by fusion between the respective spinous processes and transverse processes. In this case, In such case (e.g., a posterolateral intertransverse process fusion procedure), a 4% or more collagen matrix is preferred and it may also be desirable to add a DBM product to the rehydrated collagen-growth factor composition to make an implantable

The representative rehydrated osteogenic compositions described in the foregoing examples have good physical properties, such as cohesiveness, extensibility and retention of shape that are qualitatively better than the putty-like compositions described in U.S. patent application Ser. No. 09/023,617. Unlike the new sponge-like compositions of the present invention designed for use with a spinal cage, the putty-like forms do not retain a defined shape when compressed.

### Example 3

### Surgical Kit Containing a Collagen-Growth Factor Matrix

The new compositions or devices described in Examples 1 and 2 can be included as part of a kit containing the components of the materials. Such kits are particularly useful for health care professionals in preparing the materials and compositions of the present invention immediately before use. In addition to including the component parts of the various materials and compositions described above, a kit may also include one or more containers for mixing the components, along with optional mixing devices such as stirrers or applicators. Further, such kits can include the components in sealed, pre-measured packages. The sealed packages can be sealed septically and the amounts of the components can be pre-measured in relative amounts as described elsewhere herein. Alternatively, the kit might simply contain one or more of the new osteogenic compositions or devices in hydrated, pre-shaped, ready-to-implant form, optionally, along with an applicator.

While the preferred embodiments of the invention have been shown and described, modifications thereof can be made by one skilled in the art without departing from the spirit and teachings of the invention. The embodiments described herein are exemplary only, and are not intended to be limiting. For example, in addition to dental procedures, repair of periodontal defects and spinal fusion procedures, the new osteogenic compositions, prepared by similar methods, can be used in a variety of other applications wherever there is a need to generate bone. Such applications include induction of bone formation for hip replacement operations, knee replacement operations, treatment of osteoporosis, repair of bone tumor defects, repair of cranialmaxillafacial defects, and repair of bone fractures, to name a few. It should also be appreciated that by omitting the osteogenic components, the above-described collagen-based materials could also be used for in vivo delivery of a variety of pharmaceuticals or other agents for many therapeutic applications other than osteogenic procedures. Many variations and modifications of the invention disclosed herein are possible and are within the scope of the invention. For instance, most uses of the present compositions, devices and methods focus on human applications. However, it can be readily appreciated that similar osteogenic compositions, devices and methods are applicable to a wide variety of animals, particularly mammals. Accordingly, the scope of protection is not limited by the description set out above, but is only limited by the claims which follow, that scope including all equivalents of the subject matter of the claims. The disclosures of all patents, patent applications and printed publications identified herein are incorporated by reference.

What is claimed is:

1. A method of making an osteogenic composition, the method comprising:
combining purified collagen, an osteoinductive substance, and water containing dilute acid in a dispersing assembly comprising two vessels and a reduced diameter portion, said vessels being in mutual fluid communication by way of said reduced diameter portion;
forcing said combination from vessel to vessel through said reduced diameter portion a predetermined number of times sufficient to disperse said collagen and osteoinductive substance in said water, such that said collagen is at least partially hydrated and a dispersion is obtained;
allowing said dispersion to stand for a predetermined time interval.

2. The method of claim 1 wherein said dispersing assembly comprises two syringes, each having a plunger, and said step of passing said combination from vessel to vessel comprises sequentially depressing said plungers a predetermined number of times such that said combination is subjected to physical forces sufficient to disperse said collagen and osteoinductive substance in said water, such that said collagen is at least partially hydrated and a dispersion is obtained.

3. The method of claim 1 wherein said predetermined number of passes is up to about 250.

4. The method of claim 1 wherein said step of passing said combination from vessel to vessel comprises:
passing said combination from vessel to vessel a first predetermined number of passes;
allowing said combination to stand for a first predetermined time interval;
passing said combination from vessel to vessel a second predetermined number of passes; and
allowing said combination to stand for a second predetermined time interval, such that a dispersion is obtained.

5. The method of claim 4 wherein said first predetermined number of passes is about 5-150.

6. The method of claim 4 wherein said second predetermined number of passes is about 5-150.

7. The method of claim 4 wherein said first time interval is about 30-60 minutes.

8. The method of claim 4 wherein said second time interval is at least about 12-72 hours.

9. The method of claim 1 wherein said reduced diameter portion comprises a connector and said step of forcing said combination from vessel to vessel through said reduced diameter portion includes passing said combination through said connector.

10. The method of claim 1 further comprising extruding said dispersion to provide an extrudate.

11. The method of claim 10 further comprising molding said extrudate.

12. The method of claim 10 further comprising drying said extrudate to provide a dehydrated osteogenic matrix.

13. The method of claim 12 further comprising sterilizing said dehydrated osteogenic matrix.

14. The method of claim 13 further comprising rehydrating said dehydrated osteogenic matrix.

15. The method of claim 14 further comprising mixing a bulking material with said rehydrated matrix to provide a shapeable osteogenic implant material.

16. The method of claim 15 wherein said bulking material is particulate demineralized bone matrix.

17. The method of claim 15 further comprising shaping said osteogenic implant material.

18. The method of claim 1 wherein said dispersion comprises approximately 1-8% (wt./Vol.) collagen.

19. The method of claim 1 wherein said collagen is dehydrated fibrous bovine tendon type I collagen.

20. The method of claim 1 wherein said water containing dilute acid comprises about 10 mm HCl.

21. The method of claim 1 wherein said osteoinductive substance is chosen from the group consisting of bone growth proteins, bone morphogenetic proteins 1-13, osteogenic protein-1 or 2,FGF-I or -II, TGF-beta, GDF-5,6 or 7.

22. The method of claim 1 further comprising combining a biologically active agent other than said osteoinductive substance with said collagen/osteoinductive substance, said agent chosen from the group consisting of growth factors, cartilage inducing factors, angiogenic factors, hormones, antibiotics, antiviral compounds and anticancer compounds.

23. A method of making an osteogenic composition, the method comprising:
combining a predetermined amount of purified collagen, a predetermined amount of an osteoinductive substance, and a predetermined amount of a dilute aqueous acid solution in a dispersing assembly comprising two vessels connected by a reduced diameter portion, said vessels being in mutual fluid communication;
passing said combination from vessel to vessel a predetermined number of times such that said combination is subjected to physical forces sufficient to disperse said collagen and osteoinductive substance in said water, such that said collagen is at least partially hydrated and a thickened dispersion is obtained;
allowing said thickened dispersion to stand for a second predetermined time interval, such that a thick, extrudable dispersion is obtained;
extruding said thick, extrudable dispersion to provide an extrudate; and
drying said extrudate to provide a dehydrated osteogenic matrix.

24. An osteogenic composition comprising a product of the method of claim 1.

25. The osteogenic composition of claim 24 comprising a mixture of purified type I bovine fibrillar tendon collagen and an osteoinductive substance.

26. The osteogenic composition of claim 25 further comprising an active agent other than said osteoinductive substance, said agent chosen from the group consisting of growth factors, cartilage inducing factors, angiogenic factors, hormones, antibiotics, antiviral compounds and anticancer compounds.

27. The osteogenic composition of claim 25 further comprising a bulking material combined with said mixture.

28. The osteogenic composition of claim 27 wherein said bulkng material is particulate demineralized bone matrix.

29. The osteogenic composition of claim 25 wherein said osteoinductive substance is chosen from the group consisting of bone growth proteins, bone morphogenetic proteins 1-13, osteogenic protein-1 or 2, FGF-I or II, TGF-beta, GDF-5,6 or 7.

30. A method of making an implantable osteogenic device comprising:
preparing an osteogenic composition according to the method of claim 10;
dehydrating said extrudate to yield a dehydrated osteogenic product;
rehydrating said dehydrated product;
mixing said rehydrated product with a bulking material to provide a shapeable osteogenic implant material.

31. The method of claim 30 further comprising shaping said osteogenic implant material to provide an implantable osteogenic device.

32. A shaped osteogenic device comprising a product of the method of claim 31.

33. A method of making an implantable osteogenic device comprising:
preparing an osteogemc composition according to the method of claim 10;
dehydrating said extrudate to yield a dehydrated osteogenic product;
rehydrating said dehydrated product;
inserting said rehydrated product into a spinal cage to provide an osteogenic device.

34. An osteogenic spinal cage comprising a product of the method of claim 33.

35. A method of inducing osteogenesis in a subject in need thereof comprising implanting in said subject at a site where osteogenesis is desired a device according to claim 30.

36. The method of claim 35 wherein said site is a dental or periodontal defect site.

37. A method of inducing osteogenesis in a subject in need thereof comprising implanting in said subject in the disk space between two vertebral bodies that are desired to be fused together an osteogenic spinal cage according to claim 34.

38. A kit comprising a predetermined quantity of an osteogenic composition according to claim 25 and a sterility-maintaining cover.

39. The kit of claim 38 further comprising a mixing container.

40. The kit of claim 38 further comprising a predetermined quantity of a bulking material.

41. A method of making a collagenous matrix comprising:
combining collagen and a water containing dilute acid in a dispersing assembly comprising two vessels and a reduced diameter portion, said vessels being in mutual fluid communication by way of said reduced diameter portion;
passing said combination from vessel to vessel a predetermined number of times such that said combination is forced through said reduced diameter portion sufficient to disperse said collagen in said water, such that said collagen is at least partially hydrated and a dispersion is obtained;
allowing said dispersion to stand for a predetermined time interval to yield an extrudable dispersion.

42. The method of claim 41 further comprising molding said extrudable dispersion.

43. The method of claim 41 further comprising dehydrating said dispersion.

44. The method of claim 43 further comprising rehydrating said dehydrated dispersion.

45. The method of claim 44 further comprising mixing a bulking material with said rehydrated dispersion.

46. The method of claim 41 further comprising combining a biologically active agent with said collagen and water.

47. The method of claim 41 wherein said collagen comprises about 1-8 wt % of said dispersion.

48. A collagenous matrix comprising the product of the method of claim 41.

49. A method of administering a biologically active agent to a subject in need thereof comprising:
preparing a delivery vehicle comprising the collagenous matrix of claim 41;
incorporating a biologically active agent into said delivery vehicle; and
implanting said delivery vehicle at a selected site in the body of said subject; and
allowing said biologically active agent to be released from said delivery vehicle at said site.

50. The method of claim 49 wherein said implanting comprises surgical placement of said delivery vehicle.

51. The method of claim 49 wherein said implanting comprises injecting said delivery vehicle.
